(19) United States
(12) Patent Application Publication
(10) Pub. No.: US 2016/0151017 A1
(43) Pub. Date: Jun. 2, 2016

(54) SENSOR ARRANGEMENT FOR RECORDING ELECTRIC SIGNALS

(71) Applicant: BIOTRONIK SE & CO. KG, Berlin (DE)

(72) Inventors: Marc BARTELS, Berlin (DE); Stephan FANDREY, Affoltern am Albis (CH); Jens PHILIPP, Berlin (DE)

(73) Assignee: BIOTRONIK SE & CO. KG, Berlin (DE)

(21) Appl. No.: 14/946,179

(22) Filed: Nov. 19, 2015

Related U.S. Application Data
(60) Provisional application No. 62/085,248, filed on Nov. 27, 2014.

(51) Int. Cl. A61B 5/00; A61B 5/04; A61B 7/02; A61B 5/11; A61B 8/00

(52) U.S. Cl. CPC A61B 5/6832; A61B 5/11; A61B 8/4236; A61B 7/02; A61B 5/0059; A61B 5/04

(57) ABSTRACT

Embodiments include a sensor arrangement including a primary sensor in the form of a self-adhering surface electrode that records electric signals. The self-adhering surface electrode includes a primary sensor to contact skin, and a secondary sensor, which differs from the primary sensor, to detect a secondary physical variable. In addition, the sensor arrangement includes an adapter, via which the primary sensor and the secondary sensor are interconnected to form a detachable unit.

This application claims the benefit of U.S. Provisional Patent Application 62/085,248 filed on 27 Nov. 2014, the specification of which is hereby incorporated herein by reference.

### BACKGROUND OF THE INVENTION

1. Field of the Invention

Embodiments of the invention generally relate to a sensor arrangement with a primary sensor including a self-adhering surface electrode that records electric signals.

2. Description of the Related Art

Generally, self-adhering surface electrodes for recording electric signals have a sensor side, which is intended for skin contact and which is self-adhering in order to adhere the surface electrode to skin and thus produce reliable contact with the skin.

Typically, self-adhering surface electrodes for recording electric signals in humans and animals have long been used as a matter of routine in clinical practice and in research.

For example, generally, electrocardiograms (ECGs) for cardiac activity, electromyograms (EMGs) for muscle activity, electroneurograms (ENGs) for conduction speed in nerves, and electrooculograms (EOGs) for eye movement are produced using the electrodes.

Typically, many of the electrodes are fastened on the skin of a patient using a separate adhesive film or an adhesive film already integrated around the electrode, and are then wired to an evaluation unit via a plug.

Generally, when recording electroencephalograms (EEGs) for brain activity, two-sided adhesive films are used as well as caps, to which the electrodes are fastened.

In the case of ECGs, these electrodes are typically disposable articles.

Generally, the actual active surface of the ECG electrode is often round and surrounded by a self-adhering plastic film, with or without contact gel reservoir. Typically, the contour of the film varies depending on a type from a round design or a square design to a complex geometry provided with cutouts (for example as shown in FIGS. 2A-2C herein). The actual electrode surface is typically round and has a diameter of approximately 5-8 mm.

In the case of ECGs, three to twelve electrodes are typically positioned in a defined manner around the heart and on the extremities.

Generally, besides fluoroscopy, the n-channel ECG (n>1) is the most important instrument in an electrophysiological examination (EPE) for diagnosing cardiological arrhythmias, for example in the case of catheterization and ablation.

Typically, magnetic resonance tomography (MRT), unlike most other non-ionizing modalities, provides spatially and temporally resolved soft tissue contrast and thus enables a broad spectrum of diagnostic applications for organs or anatomies that are difficult to access, such as the brain, the spinal column, the joints, the abdomen, the cardiovascular system, etc.

Generally, a combination of both medical measurement methods would make it possible to perform an EPE in the MRT as far as interventional or intraoperative MR (iMR) with the objective of performing ablations more precisely and using methods for medical imaging which are not based on ionizing radiation in order to protect the patient and hospital staff.

Typically, the direct combination of ECG and MRT poses technical challenges for physical reasons alone, in particular due to the fact that the three main components of the MRT are independent of one another and consequently may be addressed separately. For example, generally, the three main components include the strong homogenous static magnetic field in the Tesla range (1^{st}), the three magnetic gradient fields directed orthogonally to one another (2^{nd}) and the electromagnetic high-frequency fields (3^{rd}). As such, there is a need to address the disturbance-free measurement of electrophysiological signals in the environment of strong homogenous static magnetic fields, which are falsified by the magneto hydrodynamic effect (MHD). Disturbance caused by magnetic gradient and electromagnetic high-frequency fields is not discussed herein, since, generally, there are independent strategies for solving such a problem.

Typically, the magneto hydrodynamic effect (MHD effect) is caused physically by moved charge carriers in a magnetic field that are forced by the Lorentz force in a certain direction, depending on the vectorial direction of movement of the charge carriers and the orientation of the magnetic field. Generally, the MHD effect, as a pseudo-periodic electrophysiological disturbance variable in the ECG, is induced primarily by the blood flow in the aortic arch during systole. Typically, moved charge carriers in the blood are pressed against the blood vessel wall by the Lorentz force and temporarily accumulate there locally. Generally, this temporary charge density superimposes, as potential, the ECG to be recorded, in particular in the QRS complex thereof, and can be measured with the ECG on the skin surface of the patient.

### BRIEF SUMMARY OF THE INVENTION

One or more embodiments of the invention provide an arrangement that improves application of self-adhering surface electrodes and may simplify the application of self-adhering surface electrodes, compared with known devices.

At least one embodiment of the invention includes a sensor arrangement with a primary sensor, such as a self-adhering surface electrode, that records electric signals. In at least one embodiment, the primary sensor may include a sensor side that contacts skin of a patient. In one or more embodiments, the sensor arrangement may include a secondary sensor, which differs from the primary sensor, to detect a further physical variable. In one or more embodiments, the sensor arrangement may include an adapter, via which, the primary sensor and the secondary sensor are interconnected to form a detachable unit.

Embodiments of the invention enable a simple and reliable application of self-adhering surface electrodes with a secondary sensor that differs from a primary sensor to detect a further physical variable. In one or more embodiments, the adapter may include a mechanical adapter, wherein self-adhering recording electrodes may be used in combination with further sensors in contact with the patient's body.

Via the sensor arrangement, one or more embodiments may record electrocardiograms on patients in a magnetic resonance tomograph.

One or more embodiments of the invention detect an additional measured variable, which characterizes the cardiac activity but is not influenced by the MHD effect, for example via acoustic or optical secondary sensors. In at least one embodiment, the signals of the secondary sensors may be offset against signals of the primary sensor, such as an ECG electrode.

By way of one or more embodiments of the invention, the respective secondary sensors may be fastened to the body of the patient close to the respective primary sensor, for example the ECG electrode or surface electrode. As such, in at least one embodiment, each ECG channel may be separately corrected in an optimal manner.

One or more embodiments of the invention provide a stable and reproducible arrangement of the primary and secondary sensors, both relative to one another and on the skin surface of the patient. By way of at least one embodiment, clinical progress may not be influenced by the introduction of the additional secondary sensor. In one or more embodiments, the sensor arrangement may include secondary sensors that require direct body contact, such as acoustic and optical sensors. In at least one embodiment, other ECG electrodes may be used with the sensor arrangement, such as disposable electrodes.

The inventors have found that it is disadvantageous to combine the primary and secondary sensor to form a fixedly connected unit. For example, generally, the disadvantages found include wherein
it is not possible to provide a disposable version since the secondary sensor should be reusable where possible for cost reasons, and
an individual performing an examination may no longer use a favored primary electrode.

Generally, however, combining the primary and secondary sensors is not possible when the secondary sensor requires direct body contact, as is the case with acoustic and optical sensors.

With the sensor arrangement according to one or more embodiments of the invention, the secondary sensor may be attached to a suitably formed adapter or may be fixedly integrated therein. In at least one embodiment, the adapter may be combined with the self-adhering surface electrodes, to record electric signals.

In one or more embodiments, the adapter and the secondary sensor may be fixedly interconnected, and the primary sensor may be detachably connected to the adapter.

In at least one embodiment, the adapter may be detachably fastened to the primary sensor on a sensor side thereof, such that the adapter may face away from the sensor side of the primary sensor that contacts the skin of the patient.

In one or more embodiments, the adapter may include a flat adhesive portion, which may be directly fastened to the skin, and may include an adapter side that contacts the skin. In at least one embodiment, the adhesive portion may include a cutout, such that the primary sensor may be fitted on the adapter side facing away from the adapter side that contacts the skin and may directly contact the skin through the cutout.

In one or more embodiments, the cutout may be located at least approximately central in the adhesive portion.

In at least one embodiment, the adhesive portion may include a plurality of markings located around the cutout. In one or more embodiments, the plurality of markings may be concentric with the cutout and may facilitate the attachment of the primary sensor externally on the adhesive portion of the adapter in a manner centered with the cutout, such that the primary sensor contacts the skin through the cutout.

By way of at least one embodiment, the adapter may include a portion protruding laterally beyond the adhesive portion, wherein the laterally protruding portion may carry one or more of the secondary sensor and a plug connector that corresponds with the secondary sensor.

In one or more embodiments of the invention, the secondary sensor may not be fastened to the adapter centrally, but may be fastened at or in a vicinity of an edge thereof, such as peripherally. As such, in at least one embodiment, that the primary sensor may be fastened centrally on the adapter.

According to one or more embodiments, the sensor arrangement may include a plug connector that corresponds with the secondary sensor, wherein the plug connector may be electrically connected to the secondary sensor and may be fastened to the adapter. In at least one embodiment, the secondary sensor may be connected via the plug connector via cables to a device that processes the signals detected by the secondary sensor, such as a processor. In one or more embodiments, the secondary sensor may include a wireless transmitter to transmit one or more signals wirelessly.

One or more embodiments of the invention may include an adapter with a secondary sensor that may couple to a sensor arrangement, such as the sensor arrangement discussed previously. In at least one embodiment, the adapter may not include a fastened primary sensor.

In one or more embodiments, the secondary sensor may include a plurality of sensors, and may be or may include one or more of an acoustic sensor, an acceleration sensor and an optical sensor. In at least one embodiment, the secondary sensor or sensors may not be influenced, completely or partially, by a magnetic resonance tomograph. In one or more embodiments, the secondary sensor or sensors may include an ultrasonic sensor.

In at least one embodiment of the invention, the adapter may include, at least in part, a soft biocompatible plastic. In one or more embodiments, the plastic may include one or more of silicone, coated or uncoated paper, and tissue made of natural and/or plastic materials.

### BRIEF DESCRIPTION OF THE DRAWINGS

The above and other aspects, features and advantages of at least one embodiment of the invention will be more apparent from the following more particular description thereof, presented in conjunction with the following drawings, wherein:

FIG. 1 shows an electrocardiography system with a sensor arrangement according to one or more embodiments of the invention;

FIGS. 2A-2C show various prior art arrangements of self-adhering surface electrodes that record electric signals;

FIG. 3 shows a sensor arrangement according to one or more embodiments of the invention with an adapter, wherein the adapter is slipped over a primary sensor;

FIGS. 4A-4C show variants of the primary sensor fitted onto the adapter, according to one or more embodiments of the invention;

FIGS. 5A-5C show variants of the secondary sensor in contact with the adapter, according to one or more embodiments of the invention; and

FIG. 6 shows an adapter with concentric circles as a centering aid to center primary sensors that are symmetrical about a central point, according to one or more embodiments of the invention.

### DETAILED DESCRIPTION OF THE INVENTION

The following description is of the best mode presently contemplated for carrying out at least one embodiment of the invention. This description is not to be taken in a limiting sense, but is made merely for the purpose of describing the general principles of the invention. The scope of the invention should be determined with reference to the claims.

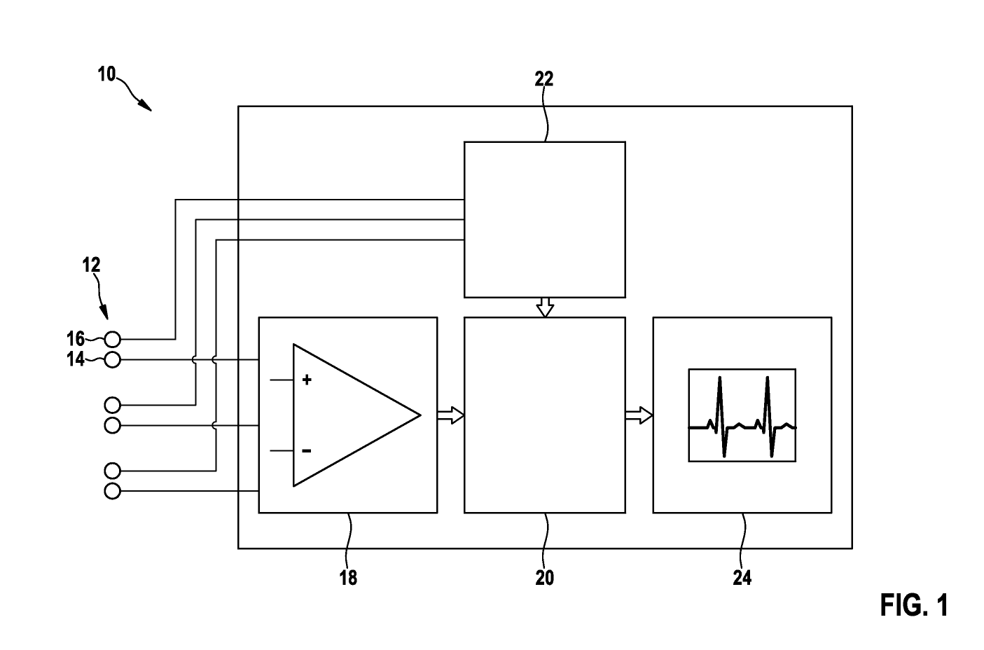

FIG. 1 shows an electrocardiography system 10 with a plurality of sensor arrangements 12 according to one or more embodiments of the invention. In at least one embodiment, each of the sensor arrangements 12 may include at least one primary sensor 14 and at least one secondary sensor 16. In one or more embodiments, the at least one secondary sensor 16 may include a self-adhering surface electrode that records electric signals, such as electrocardiograms. In at least one embodiment, the at least one primary sensor 14 may be connected to a signal processing unit 18 to filter and amplify the recorded electric signals. In one or more embodiments, the electric signals that are processed may be fed to a signal evaluation unit 20, which evaluates the recorded electric signals under consideration of secondary signals.

By way of at least one embodiment, the at least one secondary sensor 16 may receive the secondary signals and may be connected to a secondary signal processing unit 22. For example, in one or more embodiments, the at least one secondary sensor 16 may include one or more of optical sensors, acoustic sensors and motion sensors. In at least one embodiment, the secondary sensors 16 may not be influenced, completely or partially, by use in a magnetic resonance tomograph. In one or more embodiments, the secondary sensors 16, as a result following processing by the secondary sensor processing unit 22, may provide a secondary signal, on the basis of which the signal evaluation unit 18 may determine and compensate for the influence, for example of the magneto hydrodynamic effect on recorded ECG signals. As such, in at least one embodiment, the electrogram may be corrected and may then be presented, for example to a doctor, via one or more of a printer and a display 24.

FIG. 1 shows a schematic illustration of the sensor arrangements 12, wherein further details of the sensor arrangements 12 are shown in FIGS. 3 to 6, as will be discussed below.

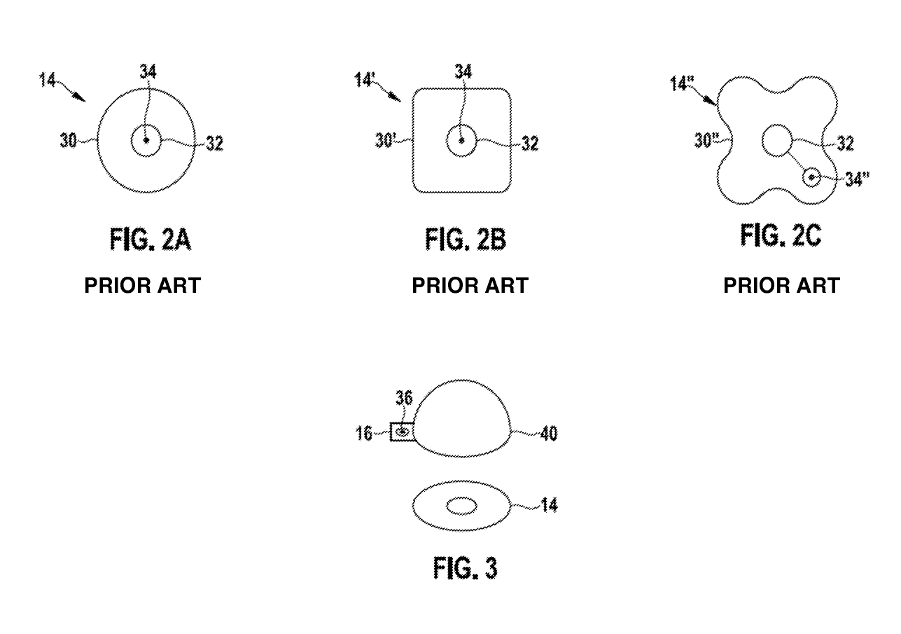

FIGS. 2A-2C show various prior art arrangements of self-adhering surface electrodes.

FIG. 2A shows a round surface electrode. As shown in FIG. 2A, the electrode includes a support 30 with adhesive surface arranged underneath. An electrode surface 32 is provided within the adhesive surface and is optionally adjacent to a contact gel reservoir, also arranged underneath. As also shown in FIG. 2A, the surface electrode 14 includes a plug connector 34, which for example may be a push button and serves to electrically connect the electrode surface 32 via a cable to a signal processing unit of an electrocardiograph.

FIGS. 2A-2C show various arrangements of the surface electrode 14, 14′ and 14″, wherein such arrangement differ by the shape of the respective support 30, 30′ and 30″. As shown in FIG. 2A, the support is round, whereas in FIG. 2B the support includes an angular design with rounded corners. FIG. 2C illustrates a support 30″ of which the contour includes cutouts for adjacent electrodes, in order to provide a large adhesive surface. As shown in FIG. 2C, the plug connector 34″ is positioned laterally.

FIG. 3 shows a sensor arrangement, according to one or more embodiments of the invention, with an adapter 40, wherein the adapter 40 may be fitted on a primary sensor, for example a surface electrode 14. In at least one embodiment, the adapter may include a secondary sensor 16 with a plug connector 36 that may be integrated in the adapter 40. In one or more embodiments, the adapter 40 may be slipped over the primary sensor 14 or may be fitted thereon, such as piggyback style, and may be held on the primary sensor 14 by integrated adhesive surfaces.

By way of at least one embodiment, the adapter 40 may include a base area, wherein at least the location at which the secondary sensor 16 is fastened to the adapter 40 may be larger than the base area, for example the support 30, of the primary electrode. In one or more embodiments, the secondary sensor 16 may be in direct contact with the skin. In at least one embodiment, for example as shown in FIG. 3, various surface electrodes 14 may include different designs and dimensions, and therefore different adapters 40 may be included accordingly.

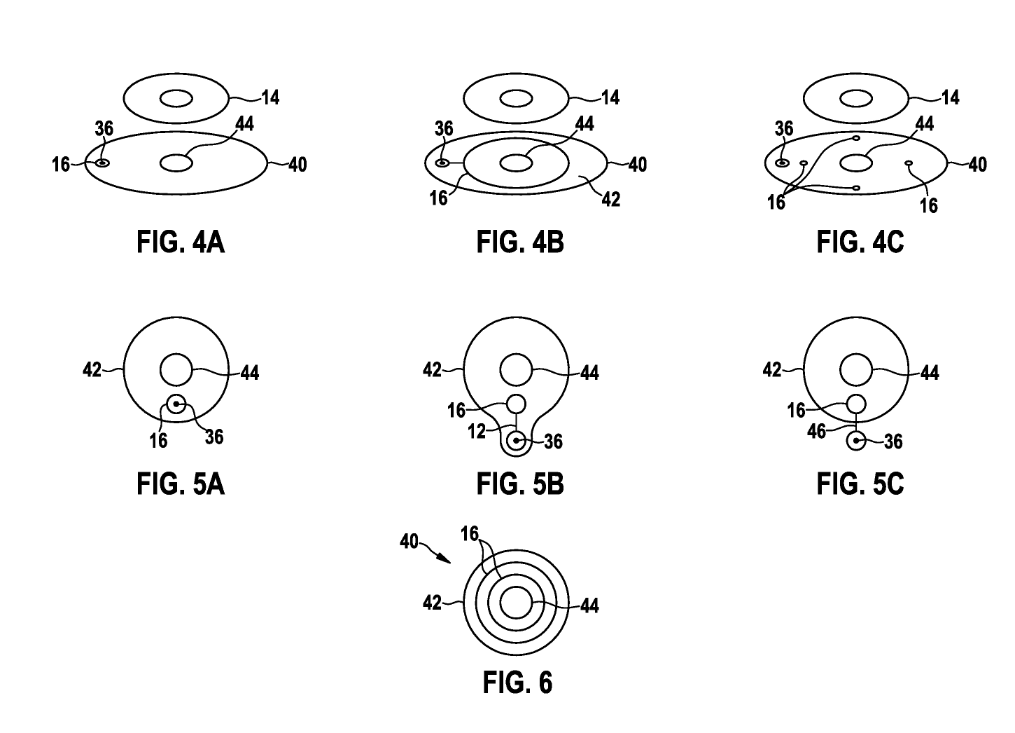

FIGS. 4A-4C show variants of the primary sensors of a sensor arrangement according to one or more embodiments of the invention, wherein a respective primary sensor is fitted onto the adapter. In at least one embodiment, the adapter 40′ and the respective secondary sensor 16 may form a unit. In one or more embodiments, the adapter 40′ may include an adhesive surface that fastens on skin and which may be located on an underside of an adhesive portion 42, such as a support of the adapter. In at least one embodiment, the secondary sensor 16 may be fixedly connected to the adhesive portion 42.

In one or more embodiments, the adhesive portion 42 may include a cutout 44, through which the primary sensor that is fitted externally onto the adhesive portion 42 may be in direct contact with the skin. In at least one embodiment, the outer side, such as the side facing away from the skin during use, of the adhesive portion 42 of the adapter 40 may be fastened to self-adhering surface electrodes. In one or more embodiments, the adhesive portion 42 may be correspondingly thin such that the primary sensor fitted on the outer side thereof is not distanced significantly from the skin.

The sub-variants of the sensor arrangement shown in FIGS. 4A-4C, according to one or more embodiments of the invention, differ by the different arrangement of the respective secondary sensor 16 with an associated plug connector 36. In at least one embodiment, as shown in FIG. 4A, the secondary sensor 16 may be radially offset in relation to the cutout 44 and therefore in relation to the primary sensor 14. In one or more embodiments, as shown in FIG. 4B, the secondary sensor 16 may be concentric with the cutout 44, wherein the secondary sensor plug connector 36 may be laterally offset such that the primary sensor 14 may be attached centrally relative to the cutout 44.

According to one or more embodiments, as shown in FIG. 4C, the secondary sensor 16 may include a secondary sensor array. In at least one embodiment, the secondary sensor array may be located uniformly around the cutout 44 and may include a secondary sensor plug connector 36. In one or more embodiments, the secondary sensor plug connector 36 may be laterally offset from the edge of the adhesive portion 42 of the adapter 40.

FIGS. 5A-5C show variants of the secondary sensor in contact with the adapter, according to one or more embodiments of the invention. For example, by way of at least one embodiment, FIGS. 5A-5C show variants of the plug connector 36 of the secondary sensor 16 on the adapter 40.

In one or more embodiments, as shown in FIG. 5A, the plug connector 36 of the secondary sensor 16 may be located directly above the secondary sensor 16.

In at least one embodiment, as shown in FIG. 5B, the plug connector 36 of the secondary sensor may be provided on a lateral protrusion of the adhesive portion 42, such that the plug connector 36 may be laterally offset in relation to the secondary sensor 16.

In one or more embodiments, as shown in FIG. 5C, the plug connector 36 of the secondary sensor may not be arranged at all on the adhesive portion 42, but may be connected via a cable 46 to the primary sensor 16.

FIG. 6 shows an adapter with concentric circles as a centering aid to center primary sensors that are symmetrical about a central point, according to one or more embodiments of the invention. As shown in FIG. 6, in at least one embodiment, the adhesive portion 42 of the adapter 40 may include markings 48, such as concentric rings, around the central cutout 44. In one or more embodiments, the markings 48 may be a centering aid to fit the self-adhesive surface electrodes as primary sensors.

In at least one embodiment, the primary sensor, for example the ECG electrode, may be fastened on the adapter, such as by integrated adhesive surfaces. In one or more embodiments, the adapter may include an integrated window, such as a “viewing window”, through which the electrode of the primary sensor may contact the skin.

By way of at least one embodiment, the adapter, on the underside thereof, may include an adhesive surface, such that the combination of the adapter and the electrodes may be fastened to the skin.

As such, according to one or more embodiments, the primary electrode may include an arbitrary contour, such that the individual performing the examination may continue to use a preferred ECG electrode.

At least one embodiment of the invention may include a method including one or more of:
providing the primary sensor, for example the ECG electrode, as a sterile-packed electrode;
providing the adapter with the secondary sensor, such as a disinfected and sterile adapter;
fastening the adapter to the body of the patient, wherein a number of adapters may be included to correspond to a number of ECG recording channels, for example as points of attachment, such that the cutouts in the adapters are at the desired locations of the ECG recordings;
wiring the secondary sensors, located in the adapters, to an evaluation unit thereof;
fastening the primary sensors (for example disposable ECG electrodes) on adapters fastened to the body;
wiring the primary sensors to the evaluation unit thereof;
examining the patient;
removing the wiring;
disposing of the disposable ECG electrodes; and,
providing the adapter and secondary sensor as preparation for the next procedure, for example by removing the adhesive film and disinfecting the adapter and secondary sensor.

According to one or more embodiments, the sensor arrangement provides one or more of the following advantages:
the clinical sequence of the procedure may be minimally changed;
various different ECG electrodes may be used;
disposable articles may be used; and,
various connections between the secondary sensor and the plug connector may be used, for example as a “one size fits all” arrangement.

By way of at least one embodiment, the adapter simplifies the use of the secondary sensors in the arrangement. In one or more embodiments, electrocardiography systems that require such a secondary sensor may be operated more easily. By way of at least one embodiment, electrocardiography systems may include one or more of an MRT-compatible ECG recording apparatus, and an MRT-compatible system that enables electrophysiological examinations.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE SIGNS

10 electrocardiography system
12 sensor arrangement
14 primary sensor
16 secondary sensor
18 signal processing unit
20 signal evaluation unit
22 secondary signal processing unit
24 display/printer
30 support
32 electrode surface
34 plug connector of the primary sensor
36 plug connector of the secondary sensor
50 adapter
42 adhesive portion
44 cutout
46 cable
48 markings

What is claimed is:

1. A sensor arrangement comprising:
a primary sensor with a sensor side configured to contact skin, wherein the primary sensor comprises
a self-adhering surface electrode, or
a surface electrode with an adhesive, or
a surface electrode with one or more of a fastening material and fastening mechanism,
wherein, via the sensor side, the primary sensor is configured to record electric signals;
a secondary sensor,
wherein the secondary sensor differs from the primary sensor, and,
wherein the secondary sensor is configured to detect a physical variable; and,
an adapter, wherein via the adapter, the primary sensor and the secondary sensor are
interconnected to form a detachable unit.

2. The sensor arrangement according to claim 1, wherein the adapter and the secondary sensor are fixedly interconnected, and wherein the primary sensor is detachably connected to the adapter.

3. The sensor arrangement according to claim 1, wherein the adapter is configured to be detachably fastened to the primary sensor on the sensor side that is configured to contact skin, such that the adapter faces away from the sensor side that is configured to contact skin.

4. The sensor arrangement according to claim 1, wherein the adapter comprises
a flat adhesive portion, wherein the flat adhesive portion is configured to directly fasten to the skin, and
an adapter side configured to be contacted by the skin,
wherein the adhesive portion comprises a cutout, such that the primary sensor is fitted to the adapter side facing away from the adapter side that is configured to contact skin and such that the adhesive portion is configured to directly contact the skin through the cutout.

5. The sensor arrangement according to claim 4, wherein the cutout is located at least approximately central in the adhesive portion.

6. The sensor arrangement according to claim 4, wherein the adhesive portion further comprises a plurality of markings, wherein the plurality of markings are located around the cutout and are concentric with the cutout.

7. The sensor arrangement according to claim 4, wherein the adapter further comprises a portion protruding laterally beyond the adhesive portion, wherein said portion that is laterally protruding carries one or more of the secondary sensor and a plug connector that corresponds with the secondary sensor.

8. The sensor arrangement according to claim 1, wherein the secondary sensor is fastened to the adapter at or around an edge of the adapter.

9. The sensor arrangement according to claim 1, further comprising a plug connector that corresponds with the secondary sensor, wherein the plug connector is electrically connected to the secondary sensor and is fastened to the adapter.

10. The sensor arrangement according to claim 1 further comprising:
an adapter configured to couple with a secondary sensor of said sensor arrangement.

11. The sensor arrangement according to claim 10, wherein the secondary sensor comprises one or more of an acoustic sensor, an acceleration sensor and an optical sensor.

12. The sensor arrangement according to claim 10, wherein the secondary sensor comprises an ultrasonic sensor.

13. The sensor arrangement according to claim 10, wherein the adapter comprises, at least in part, a soft biocompatible plastic.
